(19) United States
(12) Patent Application Publication
(10) Pub. No.: US 2023/0330126 A1
(43) Pub. Date: Oct. 19, 2023

(54) MODIFIED ADENOSINE NUCLEOSIDE FOR USE IN THE TREATMENT OF VIRAL INFECTIONS

(71) Applicant: Giampietro IACHETTINI, Roma (IT)

(72) Inventor: Giampietro IACHETTINI, Roma (IT)

(21) Appl. No.: 18/003,984

(22) PCT Filed: Jun. 28, 2021
(86) PCT No.: PCT/IB2021/055758

§ 371 Date: Dec. 30, 2022

(30) Foreign Application Priority Data
Jul. 1, 2020 (IT) 102020000015817
Jul. 1, 2020 (IT) 102020000015820

(51) Int. Cl. A61K 31/7076; A61K 31/708; A61K 31/522; A61K 9/20; A61K 9/48; A61K 9/16; A61K 9/46; A61K 9/08

(52) U.S. Cl. CPC A61K 31/7076; A61K 31/708; A61K 31/522; A61K 9/2013; A61K 9/2054; A61K 9/2027; A61K 9/2009; A61K 9/4858; A61K 9/4816; A61K 9/1617; A61K 9/1635; A61K 9/1623; A61K 9/1652; A61K 9/1641; A61K 9/0007; A61K 9/08

(57) ABSTRACT

An antiviral treatment of infections from Coronavirus, in particular COVID-19, is by administration of a modified nucleoside, derived from adenosine, individually or in combination with other therapeutically active substances. In particular, 3′-deoxyadenosine, or cordycepin, is administered for the treatment of a viral syndrome from Coronavirus, in particular COVID-19, in which 3′-deoxyadenosine is administered individually or in combination with at least one inhibitor or antagonist of the adenosine receptors A1 and A3 and possibly agonist of the adenosine receptors A₂ₐ and/or A_{2b}. The administration of 3′-deoxyadenosine is subsequent or simultaneous to the administration of the inhibitor, preferably inosine, a molecule which expresses both these functions.

### TECHNICAL FIELD OF THE INVENTION

The present invention relates to the antiviral treatment of infections from COVID-19 by means of the administration of a modified nucleoside, derived from adenosine, individually or in combination with other therapeutically active substances.

### Background Art

On Mar. 11, 2020, after assessing the severity levels and global spread of infection from SARS-CoV-2, the World Health Organization, WHO, declared a pandemic. This infection can lead to the development of a disease which has been referred to as COVID-19 (COrona VIrus Disease 2019). Patients experience symptoms such as: fever, fatigue, dry cough and difficulty breathing. In the most serious cases, often found in subjects already burdened by previous diseases, pneumonia, acute renal failure, cardiac events, and even death develop. Therefore, the disease induced by this virus causes systemic effects with a strong involvement of the immune system.

The present inventor has realized that the pathogenicity of Coronaviruses increases in proportion to the need for ADENOSINE expressed by the virus itself.

Therefore the origin of the disease, or pathogenesis, is to be traced back to an essential phase which precedes the protein synthesis of the viral genes: polyadenylation.

The possibility of varying the length of the gene region defined as the poly-A tail (ADENOSINE) is a peculiarity of Coronaviruses which, through this ability, can increase or decrease the efficiency of viral replication at will. Precisely this moment in the maturation of viral mRNAs can require such a large amount of adenosine triphosphate or ATP that it is capable of compromising the physiological concentration thereof, that of cyclic AMP and the levels of an essential nucleoside for normal cellular activity: ADENOSINE, and therefore of all the molecules which derive therefrom.

More in detail, Coronaviruses possess a genome of large size, consisting of a single positive RNA strand. Therefore, this nucleic acid could be directly used for protein synthesis; but it is precisely the enormous extension thereof which prevents, at least in part, this process from occurring directly.

For this reason the gene loci are included in sequences defined ORF or OPEN READING FRAME which, transcribed by the cellular RNA polymerase, generate sub-genomic fragments or MINUS STRANDS with a negative orientation.

These filaments in turn are reverse-transcribed by a polymerase of viral origin (RNA-dependent Reverse Transcriptase Polymerase or RdRP) obtained through a process referred to as FRAME SHIFTING, in sequences with a positive orientation (FIG. 1).

Each of these messenger RNAs undergoes the normal maturation process, thus it is provided with a structure referred to as CAP in position 5′ and a poly A tail in position 3′.

The poly A tail is a nucleic acid portion, simply consisting of a long sequence of ADENOSINE nucleotides, which serves as a support and binding site for the Poly A Binding Protein(s) or PABPs. These proteins are essential to the translation process, or protein synthesis, in ribosomes. They increase the stability of each messenger RNA, allowing it to be closed in a circular structure which prevents the enzymatic degradation thereof.

Coronaviruses have the peculiar ability to vary the length of the poly A tail. This allows them to modify the degree of efficiency of protein synthesis. A larger poly A tail allows to bind a higher number of PABPs, thus obtaining a more efficient translation of viral genes.

During the polyadenylation process, the poly A polymerase enzyme, or PAP, uses ATP as donor molecules of the single ADENOSINE nucleotides required for the polymerization of the poly A tail (FIG. 2).

The present inventor has realized that the very use of ATP, therefore the need for ADENOSINE in this phase, can become so enormous as to represent the very origin of the complex pathological picture induced by Coronaviruses.

In fact, using ATP, the poly A polymerase removes it from adenylate cyclase, the central enzyme in the functioning of metabotropic receptors or receptors coupled to G proteins, thus affecting the functioning thereof to various degrees. This enzyme converts ATP into cyclic AMP or cAMP. Therefore, the polyadenylation step of the viral genes using ATP is capable of decreasing the intracellular concentration of a second essential messenger such as cyclic AMP very directly and rapidly. This parameter alone is capable of systemically influencing the functioning of the whole organism (FIG. 3).

Metabotropic receptors are membrane or surface receptors, therefore, the specific part for the recognition of the ligands thereof is located outside the cell. For this reason, the molecules which use them, including 80% of the hormones of neuromodulators, etc., need second intracellular messengers which are therefore the same for thousands of ligands.

Cyclic AMP is the most important of these molecules.

When the virus decides to increase the viral load inside a host organism or in any case to maintain it at a high degree, it must generate a conspicuous viral progeny, therefore it must increase the protein synthesis efficiency. It is precisely in response to these premises that the virus increases, or maintains high, the length of the poly A tail. This occurs for every gene or group of viral genes in all the billions of viruses in replication. It is in these phases that the use of ATP can be so large and so rapid as to drastically reduce the level of cyclic AMP, compromising the functioning of the metabotropic receptors.

Thereby viral replication, causing the level of a second messenger common to all the thousands of ligands which use these receptors to decrease, manages to simulate a systemic inhibitory stimulus on the enzyme adenylate cyclase. Therefore, the polyadenylation process manages to create, directly at the intracellular level, the same microenvironment which would be obtained from a simultaneous stimulation of all the receptors coupled to inhibitory G proteins, and simultaneously prevent the function of those stimulating Gs, again due to lack of substrate, obviously ATP (FIG. 3).

All the molecules using the metabotropic receptors are coupled to both of these proteins simultaneously. Therefore, they manage to induce opposite effects on the intracellular concentration of cyclic AMP. But it is exactly the level of this molecule which exerts diametrically opposite effects which have repercussions at the systemic level.

To understand the origin of the symptoms, only the most significant ligands which entrust the transduction of the signal thereof to metabotropic receptors will be mentioned, such as: the HISTAMINE, LEUKOTRIENE-PROSTAGLANDIN-THROMBOXANE axis, ANGIOTENSIN, VASOPRESSIN, ADRENALINE and ACETYLCHOLINE, ADENOSINE, DOPAMINE, SEROTONIN, GABA, etc. It is for this reason that the pathological picture of COVID-19 patients can be exactly the sum of all the effects which these molecules would induce by acting on the corresponding receptors associated with inhibitory G subunits; thus respectively: vasoconstriction and bronchoconstriction, pro-inflammatory state with cytokine release, platelet aggregation with venous thrombosis/pulmonary thrombosis, edema, cardiac events, akinesia etc. The gustducin receptor and the olfactory receptors are also coupled to G proteins, the loss of the sense of taste and the condition of anosmia are precisely attributable thereto. Glucagon is the reason for the condition of diabetes in COVID-19 patients and for the involvement of the pancreas, rhodopsin is responsible for night vision and for the involvement of sight: all molecules which act by means of metabotropic receptors. Therefore, the decrease in the intracellular concentration of cyclic AMP is the main cause of the pathological conditions induced by Coronaviruses. For this reason, the affected organs correspond exactly to the target organs of the molecules listed above: lungs, cells of the immune system, heart, kidneys, central nervous system, pancreas, eyes, etc.

Therefore a first part of the symptoms, the most significant ones, depends on the massive use of ATP; while, obviously, the simultaneous need for adenosine reduces or prevents the synthesis of all the substrates, enzymes and metabolites formed by this nucleoside: thus NAD, NAD 5 reductase, FAD, etc., and is capable of conditioning the concentration of the inosine itself. Therefore, the polyadenylation phase of the viral genes represents the reason for the entire pathogenesis.

Blocking the polymerization of the poly A tail means preventing the binding of any viral RNA with the PABPs. Thereby, we can stop the translation of viral genes into proteins, making the assembly of the viral progeny impossible, hence the replication and spread of the virus.

This can be achieved using an adenosine nucleoside chain terminator, cordycepin or 3′-deoxyadenosine.

The ability of this molecule to cause the interruption of the synthesis of the poly A tail is known, but it is the essential role of the PABPs during protein synthesis which allows the possibility of causing a decisive antiviral function.

The poly A tail serves as a support for these proteins located at the 3′ end of each mRNA. This allows the closure of each of these filaments in a circular structure, which allows the interaction thereof with the translation initiation complex or eukaryotic translation Initiation Factor eIF4F bound to the CAP located in position 5′ (FIG. 4).

The eIF4F complex consists of three proteins: eIF4E which binds the CAP, eIF4A which has the function of helicase and eIF4G which unites them and is responsible for binding to the PABPs.

The eIF4G actively recognizes, recruits, the eIF3 proteins of the complex referred to as 43S, which is bound to the minor ribosomal subunit (40s) causing the approach thereof to the translation site in the correct position of the mRNA itself. The remainder of the 43S system consists of a ternary complex consisting of the eLF2 molecule of GTP and a methionine-tRNA-initiator. For this reason, the protein translation is defined as CAP dependent, because these molecules seem to apply an essential role alone. Instead, it is the bond with the PABPs which, by determining a conformational change in the eIF4F complex, manages to improve the functions of all the proteins forming it. Therefore, in summary, binding with the PABPs causes a greater ease in the recognition phase between mRNA and ribosomes, increases the unwinding activity of the filament (helicase), and causes a greater propensity to initiate protein synthesis.

It is for all these reasons that the PABPs manage to increase the translation efficiency of the viral and cellular genes. Obviously, all these protein complexes are influenced by further control systems. Administering cordycepin means causing a progressive, albeit casual, shortening of the poly A tail. This causes the decrease in the efficiency of the protein synthesis of the viral genes, up to the total elimination of the PABPs, therefore to the loss of the circular structure of the mRNA which undergoes a rapid enzymatic degradation.

A messenger RNA devoid of PABPs is recognized as a strand which has already completed the translation phase of the corresponding proteins, or a nonsense mRNA, thus it will never be translated by the ribosomes.

The shortening of the poly A tail is a mechanism which cells recognize as their own; the absence of PABPs causes the end of the translation of a gene. Cordycepin causes the interruption of the polymerization of the poly A tail, but it is the absence of the PABPs which determines the antiviral action.

But 3′-deoxyadenosine is also an adenosine molecule and therefore also acts by means of metabotropic receptors. The use thereof by activating the receptors A1 and A3 for adenosine, associated with Gi proteins (inhibitory G), induces the same effect caused by viral replication, amplifying the symptoms. The inventor of the present invention has therefore found that such a problem can be overcome by administering an inhibitor or antagonist of the receptors A1 and A3 and preferably an agonist of the adenosine receptors A₂ₐ and/or A_{2b}.

In the light of the previous considerations, the administration of Adenosine has been tried in clinics, however if such an administration has proved useful in some patients, in others it has complicated the clinical picture.

One explanation may lie in the fact that the receptors A1 for Adenosine alone mediate these effects, obviously caused by the decrease in cyclic AMP:
bronchial constriction
vasoconstriction
platelet aggregation
atrioventricular block
inflammation with release of interleukin 6 (IL-6).

### SUMMARY OF THE INVENTION

The present inventor has surprisingly discovered that, by administering 3′-deoxyadenosine or Cordycepin, in combination with at least one inhibitor of the adenosine receptors A1 and A3, it is possible to obtain an antiviral effect without inducing the side effects induced by adenosine or an adenosine molecule.

Therefore, the present invention relates to 3′-deoxyadenosine, configurational isomers, diastereosiomers, enantiomers thereof, racemes or mixtures thereof or salts with pharmaceutically acceptable acids, for use in the treatment of a viral or bacterial syndrome in which, for the translation of the genes of these pathogens, the messenger RNAs thereof are added with a poly A tail and therefore with the related PABPs, and in which the partial or total absence of this/these proteins reduces the efficiency of the translation or protein synthesis of the genes thereof, to the point of preventing the replication thereof, in which the virus is a Coronavirus, in particular COVID-19, an orthomyxovirus, a picornavirus, in particular poliovirus, or a togaviridae, and wherein the bacterium is malaria plasmodium, in which the 3′-deoxyadenosine is administered individually or in combination with at least one inhibitor or antagonist of the adenosine receptors A1 and A3, in which, when 3′-deoxyadenosine is administered in combination with said inhibitor, the administration of 3′-deoxyadenosine is subsequent or simultaneous to the administration of said inhibitor.

The invention further relates to 3′-deoxyadenosine for use in the treatment of a viral syndrome from Coronavirus, in particular COVID-19, in which the 3′-deoxyadenosine is administered in combination with at least one agonist of the adenosine receptors A₂ₐ and/or A_{2b} and in which the administration of 3′-deoxyadenosine is subsequent or simultaneous to the administration of said adenosine receptor agonist.

The invention further relates to pharmaceutical compositions comprising 3′-deoxyadenosine and an inhibitor or antagonist of the adenosine receptors A1 and A3 and agonist of the receptors A₂ₐ and/or A2b, preferably inosine (or hypoxanthine), a molecule which expresses both of these functions.

The invention also relates to 3′-deoxyadenosine and inosine for use in the preventive or therapeutic treatment of a viral or bacterial syndrome, preferably a viral syndrome from Coronavirus, in particular COVID-19, orthomyxovirus, picornavirus, in particular poliovirus, or a togaviridae, and in which the bacterium is malaria plasmodium, in which 3′-deoxyadenosine and inosine are optionally administered with Adenosine.

The invention also relates to inosine administered individually or in combination, separately or jointly, with adenosine.

These and further objects, as outlined in the appended claims, will be described in the following description. The text of the claims must be considered as included in the description for the purpose of assessing the sufficiency of the description.

In the following description, the phrase “administered in combination” or “administration in combination” means an administration of two or more active substances separately (i.e., in different dosage units), in rapid succession or with a time interval between one administration and the subsequent one, or an administration of two or more active ingredients jointly, i.e., in the same dosage unit.

Further features and advantages of the invention will become apparent from the following description of preferred embodiments thereof, given by way of indicative and non-limiting examples.

### BRIEF DESCRIPTION OF THE DRAWINGS

FIGS. 1-2 describe the replication mechanisms of the SARS COV-2 coronavirus and the polyadenylation process;

FIG. 3 describes a part of the signaling pathways of the metabotropic adenosine receptors;

FIG. 4 describes the circular shape assumed by the protein-mRNA complex;

FIGS. 5-6 illustrate the operation of Cordycepin;

FIG. 7 describes the role of inosine;

FIG. 8 describes the competitive inhibition mechanism established by the administration of inosine;

FIG. 9 describes the entire signaling pathways of the adenosine receptors;

FIG. 10 depicts the nucleic acid of orthomyxoviruses, and the transcription mechanisms.

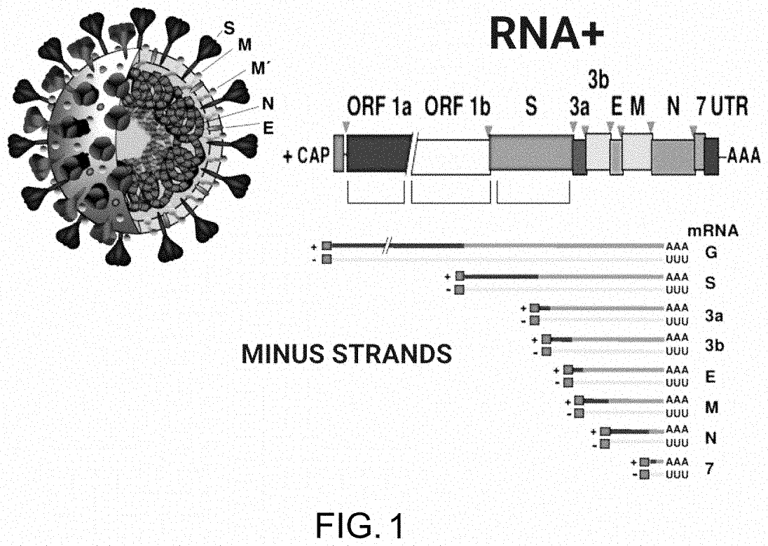

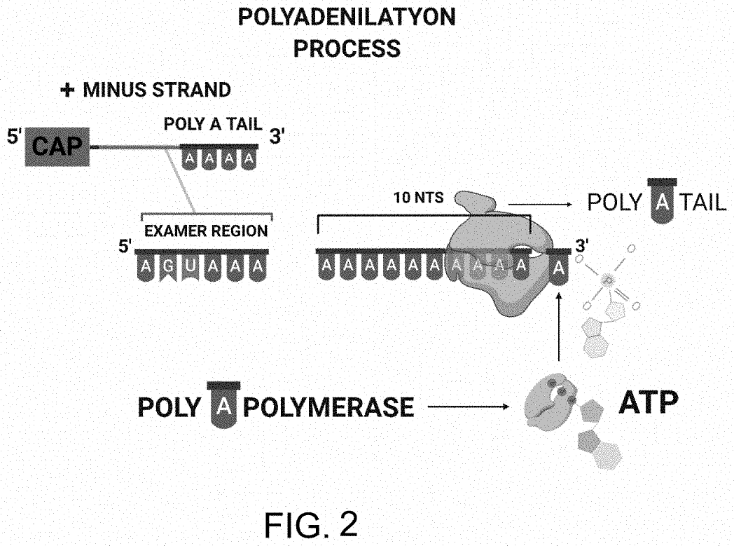

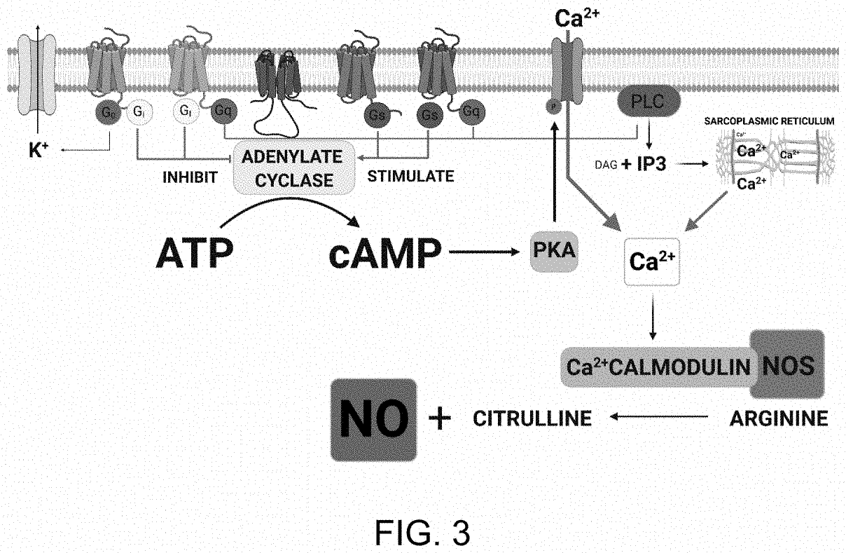

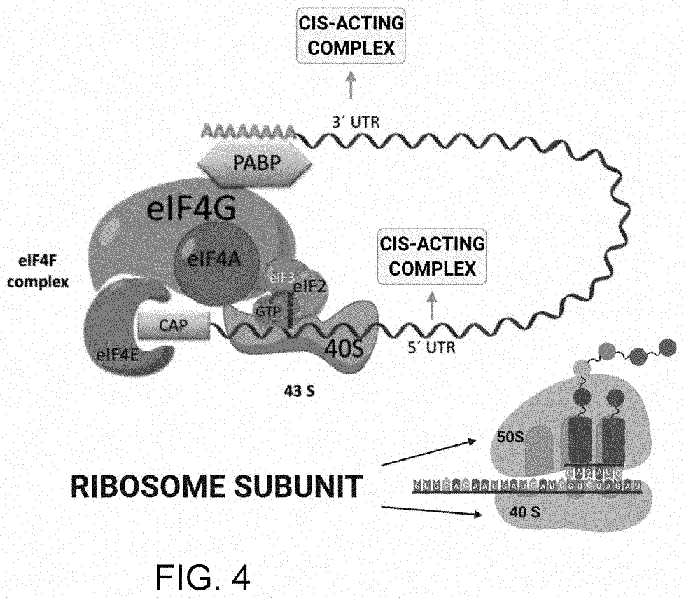

### DETAILED DESCRIPTION OF THE INVENTION

According to a first aspect, the present invention relates to 3′-deoxyadenosine, configurational isomers, diastereoisomers, enantiomers thereof, racemes or mixtures thereof or salts with pharmaceutically acceptable acids, for use in the treatment of a viral or bacterial syndrome in which, for the translation of the genes of these pathogens, the messenger RNAs thereof are added with a poly A tail and therefore with the related PABPs, and in which the partial or total absence of this/these proteins reduces the efficiency of the translation or protein synthesis of the genes thereof, to the point of preventing the replication thereof, in which the virus is a Coronavirus, in particular COVID-19, an orthomyxovirus, a picornavirus, in particular poliovirus, or a togaviridae, and in which the bacterium is malaria plasmodium, in which the 3′-deoxyadenosine is administered individually or in combination with at least one inhibitor or antagonist of the adenosine receptors A1 and A3 and possibly with an agonist of the adenosine receptors A₂ₐ and/or A_{2b} and in which, when the 3′-deoxyadenosine is administered in combination with said inhibitor and/or with said agonist, the administration of 3′-deoxyadenosine is subsequent or simultaneous to the administration of at least one adenosine receptor inhibitor.

3′-deoxyadenosine (or Cordycepin) has the following chemical structure:

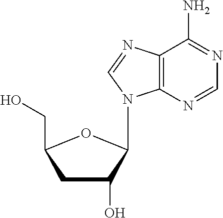

It is a derivative of the nucleoside adenosine, different therefrom due to the absence of an oxygen atom in the 3′ position of the ribose ring. Cordycepin is an isolated molecule in the fungus cordiceps sinensis or caterpillar, but it can also be obtained synthetically. Guanosine is found in this fungus, which manages to partially simulate the effect of nitric oxide by increasing the level of GTP, adenosine and another modified nucleoside 3′-deoxyuracil, which contributes to the shortening of the poly A tail in the first transcription step of mRNA strands with negative orientation. All these molecules are to be considered adjuvants of the therapy described here.

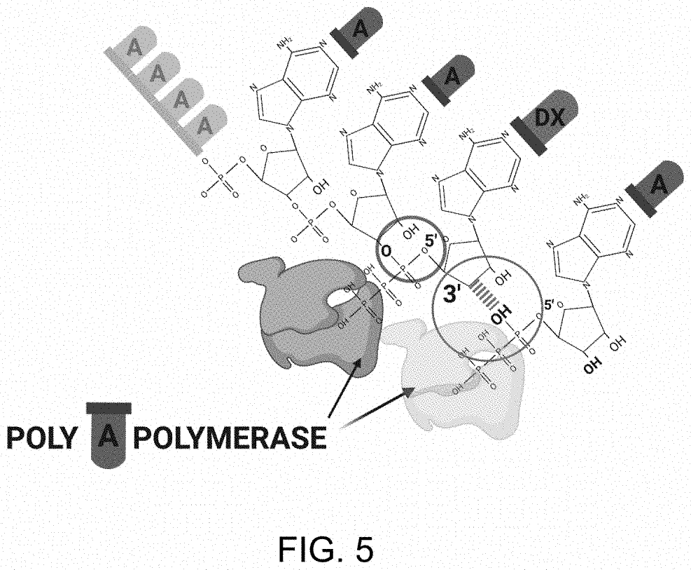

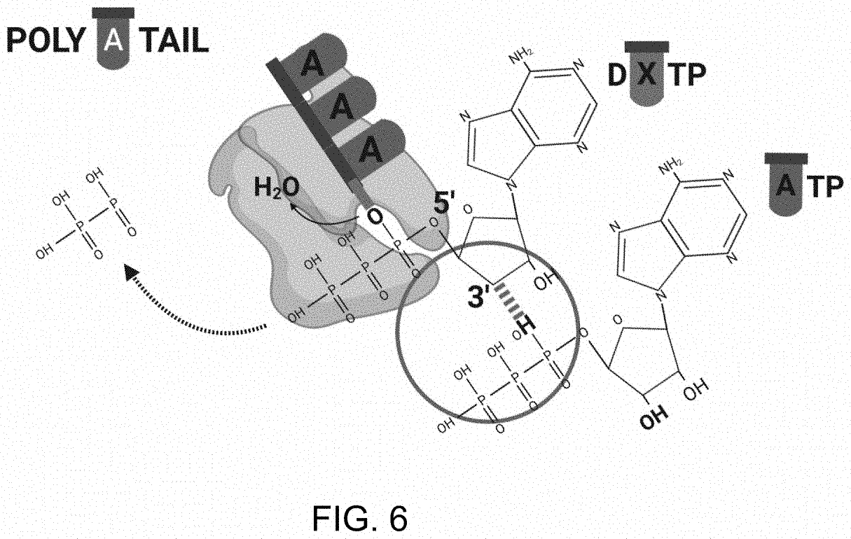

The mere absence of oxygen in the 3′ position of the ribose gives it the definition of a chain terminating molecule, as this feature can prevent the formation of the phosphodiester bond in this position of the molecule. The blocking of the polymerization of the poly A tail does not allow the binding between the viral mRNAs and the PABPs, essential molecule for protein synthesis, therefore for the translation of viral genes into proteins; this prevents the synthesis of structural and non-structural viral proteins, therefore the assembly of the viral progeny, bloccking the sp^{r}ead of the virus. The cordycepin is recognized inside the cells as a normal adenosine thus it is activated, i.e., phosphorylated, and will form ATP with a small defect (DXTP or deoxy adenosine triphosphate). The presence of hydrogen in the hydroxyl group of the phosphorylated part in the 5′ position of this molecule allows the DXTPs to firmly bind to the poly A tail being formed (FIG. 5); while it is the absence of oxygen in position 3′ which prevents the condensation reaction, therefore, the phosphodiester bond with the subsequent adenosine, determining the block of the polymerization of the poly A tail (FIG. 6).

As the concentration of cordycepin increases, the possibility that it is one of the first adenosines to be replaced will increase proportionally. Fewer than a few tens of adenine nucleotides will prevent binding to even a single PABP. At this point a 3′-deoxyadenosine molecule will block the translation of a viral gene.

3′-deoxyadenosine has a much higher affinity for poly-A polymerase with respect to adenosine, thus it is the molecule which the enzyme will prefer to use to polymerize the poly A tail of the viral mRNAs.

Poly-A polymerase is highly specific for ATP, but is not selective for adenosine. The structure thereof allows to differentiate the purine bases, thus to select adenosine at the expense of guanosine by recognizing only the amino group, NH₂, in position 2′. But many molecules which comprise a purine or indole structure associated with the nitrogenous adenine base, therefore comprising a pyrimidine ring associated with imidazole, can be used for the polymerization of the poly A tail. Therefore many molecules or isomers which are endowed with this basic structure can express a function equivalent to cordycepin. A structure comprising an indole ring which has at least one oxygen in the 2′ position of imidazole, can be phosphorylated and function as a chain terminating molecule. The absence of ribose, which cannot be bound precisely in the 2′ position, causes the lack of the group which participates in the formation of the phosphodiester bond. Other molecules which include more complex structures, added to a basic structure attributable to an adenosine can serve to increase the half-life of the molecule. But if the function of these molecules is to allow the termination of the polymerization of the poly A tail, then they can be considered equivalent to cordycepin. This molecule has an oxygen in the hydroxyl group in position 2′, this determines the exclusive use thereof for the RNA. The absence of oxygen in the 3′ position causes the antiviral function thereof which in other modified nucleosides, such as AZT (thymine), was achieved by means of the addition of nitrogen groups, which made the molecule toxic. Therefore, cordycepin is to be considered preferable over other molecules.

The 3′-deoxyadenosine causes both the antiviral function (negativization) and the interruption of the pathogenetic mechanism. It also restores the normal levels of all the molecules consisting of adenosine, therefore NAD, FAD and inosine itself; even if the absence of oxygen in position 3′ prevents it from synthesizing the cyclic AMP itself. For this reason, in subjects with high severity or who have been in a state of evident severity for a long time, the optional administration of normal adenosine may be necessary.

In preferred embodiments the antagonistic inhibitor of the receptors A1 and A3 and agonist inhibitor of the adenosine receptors A₂ₐ and A_{2b} is the same molecule and is in particular irnosirne. Inosine has the following chemical structure:

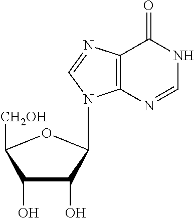

Inosine is a nucleoside consisting of a molecule of hypoxanthine bound to a ribose. Therefore, the admmi.nistration of inosine or hypoxanthine is to be considered equivalent. It is the first intermediate to have a Purine nucleus in the de novo synthesis of these nucleotides and is produced by the metabolism of adenosine.

For this reason, inosine has a fundamental role both for understanding the pathogenesis induced by Coronaviruses and in therapy; especially for the essential role thereof on the immune system. In fact, the need for adenosine by these viruses causes the decrease in the synthesis of all the substrates and enzymes consisting of this nucleoside, therefore: NAD, NAD 5 REDUCTASE, FAD, etc. and a metabolite thereof, inosine itself.

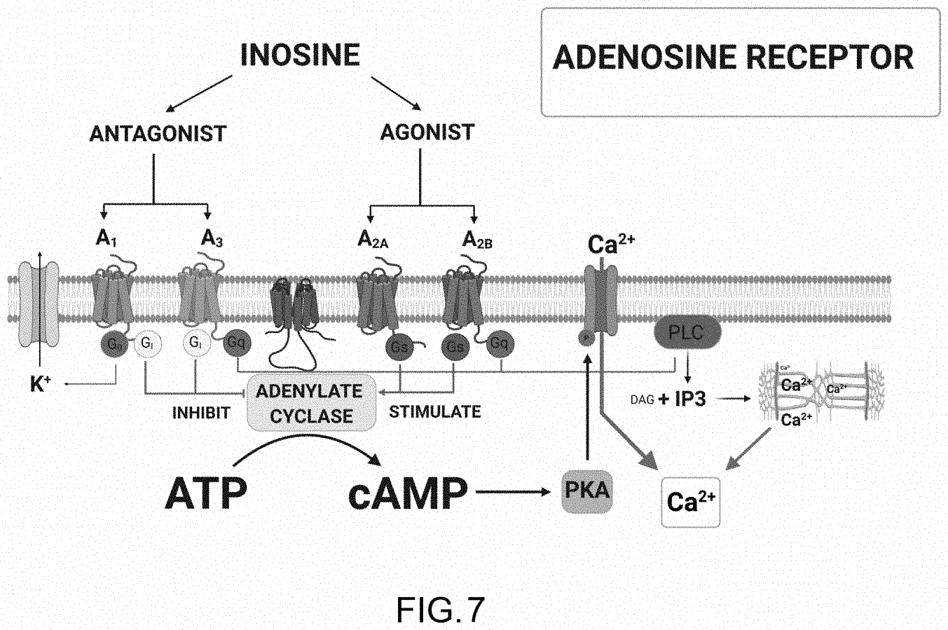

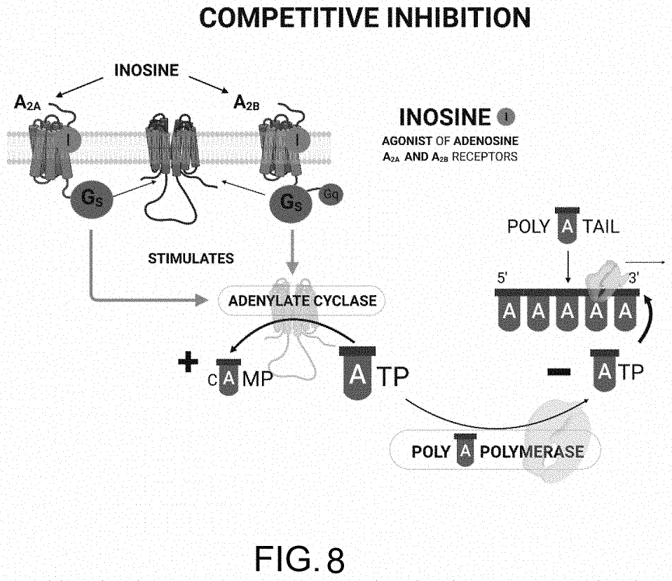

This molecule exerts a continuous stimulus on the adenosine receptors A₂ₐ and A_{2b}, therefore the physiological level thereof induces a continuous and constant increase in cyclic AMP over time, in all cells, tissues and therefore in all organs where these receptors are present (FIG. 7). This is precisely the therapeutic function thereof. Simultaneously, inosine prevents the decrease in cyclic AMP by acting as an antagonist of the receptors A1 and A3, thus eliminating the side effects of administering an adenosine molecule. It therefore has an anti-inflammatory, antiplatelet, bronchodilator, etc. role. It is the reduced synthesis of this molecule, as a consequence of the polyadenylation of the viral genes which, by reducing the stimulus on adenylate cyclase, causes the decrease of cyclic AMP; an effect which very decisively amplifies the pathological effect induced by the virus itself. Furthermore, by increasing the activity of adenylate cyclase, the administration of inosine induces a competitive inhibition mechanism between this enzyme and poly-A polymerase, competition for the substrate; thus it subtracts ATP from the forming viral poly A tail (FIG. 8).

When administered in combination, inosine and 3′-deoxyadenosine exert a synergistic effect on the adenosine receptors and complement each other in the therapeutic functions thereof.

Inosine plays an essential role especially in the cells of the immune system because it acts on an “immune check point” represented by the receptor A₂ₐ, exactly that coupled to the Gs protein alone (stimulatory G), because it obviously induces an increase in cyclic AMP. In the event of an inflammatory state, this receptor is over-expressed by cells, especially by immune cells. Therefore precisely in COVID-19 patients, this condition will significantly increase the effectiveness and speed of action of this molecule. For all these reasons, the present inventor has found that the administration of even inosine alone, or in a different embodiment of hypoxanthine, can prove effective in the treatment of a very high percentage of subjects infected with Coronavirus.

The half-life of inosine is equal to 15 hours, also this parameter, in addition to those listed, allows to obtain a therapy which can be both preventive and therapeutic.

This molecule can individually avoid the onset of a serious disease, especially if administered in the first phase of the infection and even more so in a preventive manner. It is the role thereof on the immune system which avoids the phenomenon referred to as a cytokine storm, the chronic pro-inflammatory state and platelet aggregation.

In reality, by causing a normalization of the immune system, it restores all the functions thereof, therefore both specific and non-specific immunity; this increases the number of patients who can recover, but for this reason, also obtain a negativization from the virus.

Therefore, complete therapy consists of: a properly antiviral molecule represented by a modified nucleoside and a second one which acts on the immune system. The synergistic effect between these molecules causes both healing and negativization. The optional use of adenosine causes the functioning on all patients, regardless of the initial severity conditions thereof.

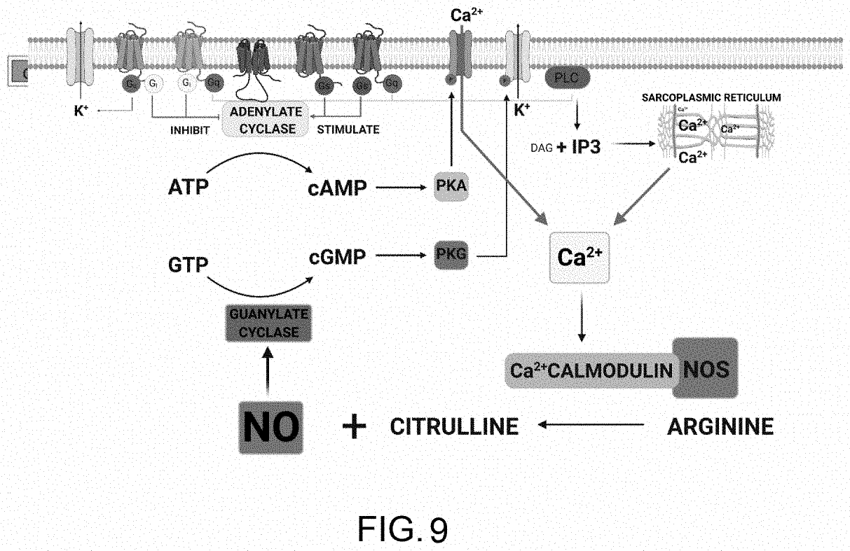

The reasons behind the pathogenesis induced by Coronaviruses involve all the molecules which are part of the signaling pathways of the metabotropic receptors. The relationships between these molecules and the enzymes consisting of adenosine describe the fundamental aspects of the disease, which can be summarized in the following main points (see also FIG. 9).

The decrease in ATP and the enzymes NAD and FAD, central to the Krebs cycle, are responsible for the condition of chronic fatigue.

The decrease in ATP also causes the condition of rigidity, or rigor, in muscle cells. In this district calcium, which initiates muscle contraction, is not recalled in the sarcoplasmic reticulum. This mechanism is contrary to the concentration gradient, thus it requires ATP to activate an ATP pump. The absence of this energetic substrate eliminates the latency phase during the different phases of muscle contraction, resulting in the condition of incomplete tetanus which characterizes the state of many COVID-19 patients.

The principle concerning nitric oxide (NO or nitrogen monoxide) is very explanatory. The problems due to a lower synthesis of cyclic AMP are added to those due to a lower synthesis of NAD on this molecule. The vasodilatory function of nitric oxide is certainly the best known feature of this molecule. The anti-inflammatory, anti-platelet aggregation properties thereof and the role thereof as a neurotransmitter make it very clear that the lower synthesis of this molecule is actually the direct cause of many of the symptoms already listed. But the synthesis reaction of the amino acid arginine requires NAD, therefore, in the most severe patients, an increase in the cyclic AMP level may not be sufficient for NO synthesis.

But nitric oxide itself is responsible for silent hypoxia, as this molecule binds hemoglobin to form meta-hemoglobin, which must not exceed 1% in the blood. This bond induces a different degree of oxidation on the ion Fe²+, preventing it from binding oxygen (just like carbon monoxide); this causes symptoms such as dyspnea, headache, and asthenia, which complete the symptomatic picture.

NADH-5-reductase is the enzyme responsible for detaching the NO molecule from the heme. The reduced synthesis of this enzyme is the cause of this condition. Vitamin C increases the activity thereof; the positive effect thereof on some COVID-19 patients is due to this.

On the other hand, the activity of the protein kinases PKA, PKG and the Ca²⁺ calmodulin kinases is instrumental in controlling the gene expression of interleukins. The activity thereof on CREB gene transcription elements, or cAMP Response Element-Binding protein, (or cAMP-sensitive elements) inhibits the activation of NF-kB and the neo-synthesis of TNF-α, IL-6 and IL-1β. Therefore, in normal concentrations, these enzymes would be able to block the positive feedback process which regulates the relationship between TNF-α, IL-1β and the release of NF-kB, which otherwise triggers a continuous self-amplification of the synthesis thereof, causing the cytokine storm.

Molecules of the Bcl-2 family are also sensitive to the level of cyclic AMP. The decrease of this second messenger increases the activity of caspases 3/9 and therefore induces apoptosis, as a consequence of viral replication. This is the cause of the permanent damage found in COVID-19 patients, to which can be added those due to the metalloproteases activated by the cytokines.

The various levels of respiratory distress (acute or ARDS) are induced by the strong inflammatory state and by the constriction of the blood vessels in the pulmonary alveoli, which damages the walls, causing edema and therefore accumulation of fluid in the lungs.

Bradykinin, which is a bronchodilator hormone which is synthesized mainly in the pulmonary alveoli, participates in this condition. This molecule acts through metabotropic receptors, but is also subject to degradation by angiotensin. This particular type of Coronavirus which causes the disease defined COVID-19 finds the binding site for spike proteins in the receptors ACE2 (Angiotensin Converting Enzyme). Unable to act, this molecule remains in the external environment and amplifies the aforementioned pathological condition.

But it is in all the disease induced by Coronaviruses that a continuous amplification of symptoms is seen. The strong redundancy of the functions of some molecules, the autocrine and paracrine modes of action and the positive feedback reactions which more strongly involve the cells of the immune system create an amplification of precise pathological mechanisms. Cyclic AMP is defined as the molecular brakes of inflammation, but also of platelet aggregation. Without this molecule, all the pathological processes listed evolve without any control, inducing a state of continuous self-amplification of the same motives at the origin of the various pathological aspects. The vital need for adenosine expressed by Coronaviruses represents the common origin of the entire pathological picture; but it represents the weak point thereof.

From all this the biochemical rationale on which the following invention is based is clear, which in a preferred embodiment includes the administration of 3′-deoxyadenosine or inosine or preferably of a combination of both, in the preventive or therapeutic treatment of the disease defined COVID-19, adapted to all Coronaviruses.

It should be noted that the absence of oxygen in the 3′ position in cordycepin does not allow the molecule to form the cyclic AMP itself. For this reason, in subjects with high severity, or who have been in a state of significant severity for a long time, adenosine can be lacking, nullifying the therapeutic effect of inosine.

Therefore, in certain embodiments, separate or joint administration of inosine in combination with adenosine or of inosine in combination with 3′-deoxyadenosine and adenosine will be included.

In certain embodiments, further administration can be included of another adenosine inhibitor of the receptors A1 and A2 and/or agonist of the adenosine receptors A₂ₐ and/or A_{2b}, such as theophylline (antagonist A1 and A3), doxofylline, 8-cyclopentyl-1,3-dimethylxanthine (antagonist A1), 8- cyclopentyl-1,3-dipropylxanthine (antagonist A1), 1-butyl-3-(3-hydroxypropyl) -8-(3-noradamantyl) xanthine (antagonist A1), caffeine (antagonist A1 and A3), 3-Et.hvl-5-benzyl-2-methyl-4-phenylethiny-6-phenyl-1, 4-(t) dihyidropyridine-3,5-dicarboxylate (antagonist A3), N-[9-Chloro-2-(2-furanyl) [1,2,4]-triazole [1,5-c]quinazolin-5-yl]benzene acetamide (antagonist A3), 1,4-Dihydro-2-methyl-6-phenyl-4-(phenyletinyl) -3.5-pyridinedicarboxylic acid, 3-ethyl-5-[(3-nitrophenyl)methyl] ester (antagonist A3), 3-Propyl-6-ethyl-5-[(ethylthio)carbonyl]-2 phenyl-4-propyl-3-pyridine carboxylate (antagonist A3), 2-phenoxy-6 (cyclohexylamino)purine hemioxalate (antagonist A3), N-[2-(2-Furanyl) -8-propyl-8H-pyrazole [4, 3- and [1,2,4]triazole[1,5-c]pyrimidine-5-yl]-N′-(4-methoxyphenyl)urea (antagonist A3), 8-Ethyl-1,4,7,8-tetrahydro-4-methyl-2-(2,3, 5-trichlorophenyl) -5H-imidazo[2,1-i]purin-5-one monohydrochloride (antagonist A3), (8R)-8-Ethyl-1,4,7,8-tetrahydro-4-5H-imidazo[2,1-i]purin-5-one hydrochloride (antagonist A3), N-(2-Methoxyphenyl) -N′-[2-(3-pyridinyl) -4-quinazolinyl]-urea (antagonist A3), 3-methoxy-4-hydroxybenzyl riboside adenine (agonist A₂ₐ), 4-(3-[6-Amino-9-(5-ethylcarbamoyl-3, 4-dihydroxy-tetrahydro-furan-2-yl) -9H-purin-2-yl]-prop-2-yl)-cyclohexanecarboxylic acid. methyl ester (agonist A₂ₐ), 3-(4-(2-((6-amino-9-((2R,3R, 4S, 5S) ) -5-(ethylcarbamoyl) -3, 4-dihydroxytetrahydrofuran-2-yl) -9H-purin-2-yl)amrino)ethyl)phenyl)propanoic acid (agonist A₂ₐ), regadenoson (agonist A2a), 2-i [6-Amino-3,5-dicyano-4-[4-4 (cyclopropylmethoxy)phenyl]-2-pyridinyl]thio]-acetamide (agonist A_{2b}), 2-Amino-4-(3-hydroxyphenyl)-6-[(1H-imidazol-2-ylmethyl) thio]-3, 5-pyridincarbonitriie (agonist A_{2b}), 2-Amino-4-(4 -methoxyphenyl) -6-[(1H-imidazol-2-ylmethyl)thio]-3,5-pyridincarbonitrile (agonist A_{2b}) and 5′-N-ethylcarboxamidoadenosine (agonist A_{2b}).

Preferred molecules are doxofylline, theophylline or caffeine.

Such molecules are known, and commercially available.

Theophylline has stimulating effects on the central nervous system, the main actions thereof are:
relaxation of the bronchial smooth muscles (bronchodilator action);
increased contractility of the heart muscle (positive inotropic action);
increase in heart rate (positive chronotropic action);
increased renal blood flow;
anti-inflammatory action (inhibition of leukotrienes);
stimulating action of the central nervous system, in particular on the medullary respiratory center (inhibition of adenosine receptors A1).

Doxofylline is a molecule with bronchodilator action, derived from theophylline, from which it differs in that it contains a dioxolane group in position 7. In animal and human studies, doxofylline showed comparable efficacy to that of theophylline, but a better tolerability profile, with significantly fewer side effects.

Doxofylline, theophylline and caffeine inhibit phosphodiesterase, so they keep the concentration of cyclic AMP high, like all phosphodiesterase inhibitor drugs.

Whether the inhibitor of the receptor A1 and A3 is administered before or simultaneously with 3′-deoxyadenosine depends on various factors, but in particular on the form of administration of the active ingredients.

If the form of administration is by injection, which will be preferred in interventions for patients in severe clinical conditions, it will be necessary to first administer the inhibitor, specifically inosine, and then the 3′-deoxyadenosine, so that the inhibitor can perform the inhibitory action thereof before the 3′-deoxyadenosine enters into circulation. Preferably, when the inhibitor is inosine, the 3′-deoxyadenosine will be injected in a time between 5 minutes and 15 hours, more preferably between 10 minutes and 6 hours, from the administration of inosine.

On the other hand, if the form of administration is oral, the inhibitor, in particular inosine, and 3′-deoxyadenosine can also be administered simultaneously, for example in the same oral formulation.

The inhibitor of the adenosine receptors A1 and A3 is preferably administered in excess by weight with respect to 3′-deoxyadenosine. Preferably, the inhibitor will be administered in a weight ratio between 1.2:1 and 2:1, more preferably between 1.2:1 and 1.7:1, with respect to 3′-deoxyadenosine.

The molecules described above are known or can be synthesized with methods known to those skilled in the art.

The invention also relates to the use of 3′-deoxyadenosine, in combination with at least one inhibitor or antagonist of the receptors A1 and A3 and agonist of the adenosine receptors A2_{A} and/or A_{2B}, more preferably inosine, for the preparation of a medicine in the form of one or more separate dosage units, for the treatment of a viral or bacterial syndrome, in which for the translation of the genes of these pathogens, the messenger RNAs thereof are added with a poly A tail and therefore of the related PABPs, and in which the partial or total absence of this/these proteins reduces the efficiency of the translation or protein synthesis of the genes thereof, up to preventing the replication thereof, in which the virus is a Coronavirus, in particular COVID-19, an orthomyxovirus, a picornavirus, in particular poliovirus, or a togaviridae, and in which the bacterium is malaria plasmodium.

The invention further relates to a formulation comprising 3′-deoxyadenosine and inosine and in which said formulation is not injectable.

In such formulations, the weight ratios are:
inhibitor, preferably inosine, between 1.2:1 and 2:1, more preferably between 1.2:1 and 1.7:1, with respect to 3′-deoxyadenosine.

In certain embodiments, the 3′-deoxyadenosine, when administered in combination with inosine, can be replaced by a different molecule chosen among:
9-hydroxyadenine,
9-(2-hydroxyethoxymethyl)adenine,
9-(4-hydroxy-3-(hydroxymethyl)butyl)adenine,
   9-(2-hydroxy-1-(hydroxymethyl) ethoxymethyl)adenine,
9-(4-acetoxy-3-(acetoxymethyl)butyl)adenine,
   9-(2-(3-methyl-2-aminobutyl)-1-(hydroxymethyl)ethoxymethyl)adenine,
9-(2-(3-methyl-2-aminobutyl)ethoxymethyl)adenine,
2-ethyl-butyl ester of (2S)-2-[(2R,3S,4R,5R)-[5-(4-aminopyrrole[2,1-f[ ] triazin-7-yl)-5-cyano-4-hydroxy-tetrahydrofuran-2-ylmethoxy]phenoxy-(S)-phosphorylamino)propionic acid,
(2S,3S, 4R, 5R) -2-(4-amino-5H-pyrrole [3,2-d]pyrimidine-7-yl) -5-(hydroxymethyl) pyrrolidine-3-hydroxy,
mono-di- or tri-phosphorylated 3′-deoxyadenosine,
   (2R,3R, 4S, 5R) -2-(6-amino-9H-purin-9-yl) -5-(4-hydroxy-3-(hydroxymethyl) but y),
(2R,3R, 4S, 5R) -2-(6-amino-9H-purin-9-yi) -5-((1, 3-dihydroxypropan-2-yl) oxyrethyl,
   (2R,3R, 4S, 5R) -2-(6-aminor-9H-p-rin-9-yl) -5(-(2-hydroxyethoxy) methyl),
(2R,3R, 43, 5R) -2--(6--amino-9-9M-purin--9-yl) --5--((2--bis (acetyloxymethyl)) ethyl)
   (2R,3R, 4S, 5R) -2-(6-armino-9H-purin-9-yl) -5-(2-amino-3-methylbutaneyloxy-2-(1 hydroxymethyl) ethoxymethyl
   (2R,3R, 4S, 5R) -2-(6-amino-9H-purin-9-yl) -5-(2-amrino-3-methylbutaneyloxy-2-ethoxymethy),
indole-3-carbinol,
configurational isomers, diastereomers, enantiomers thereof, racemes or mixtures thereof or salts thereof with pharmaceutically acceptable acids, for use in the treatment of a viral or bacterial syndrome, in which for the translation of the genes of these pathogens, the messenger RNAs thereof are added with a poly A tail and therefore with the related PABPs, and in which the partial or total absence of this/these proteins reduces the efficiency of translation or protein synthesis of the genes thereof, to the point of preventing replication.

In particular, the virus is a Coronavirus, more in particular COVID-19, an orthomyxovirus, a picornavirus, more in particular poliovirus, or a togaviridae, and in which the bacterium is malaria plasmodium.

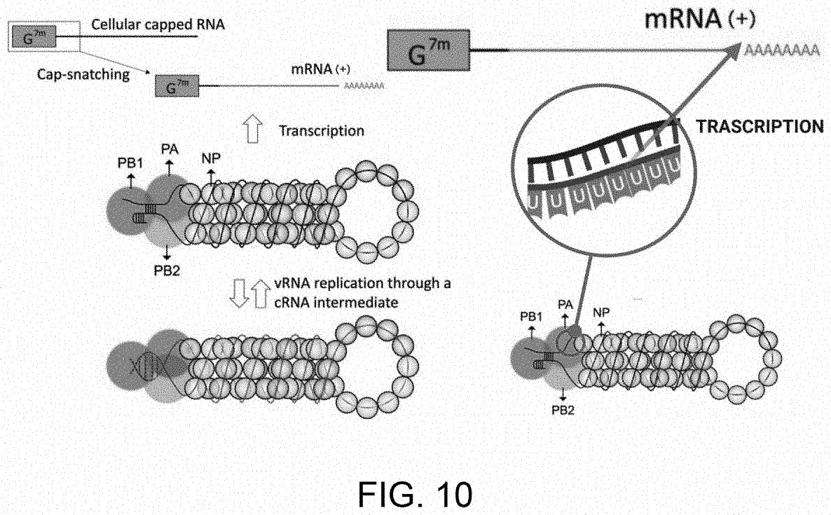

The nucleic acid of orthomyxoviruses is represented by a single strand of RNA with a negative orientation. These viruses, before protein synthesis, through a defined CAP-snatching mechanism subtract this region from cellular mRNAs, and reverse transcribe the genome thereof into a strand with a positive orientation. The region defined poly U (uracil), obviously present in the 3′ end, when reverse-transcribed, obviously forms the poly A tail (FIG. 10).

This process occurs in the nucleus, thus this region serves as a support for the nuclear PABP. The phase of exporting mRNAs from the nucleus to the cytoplasm represents a highly selective process, during which these molecules must cooperate with protein complexes which bind the CAP and the poly A tail. Nuclear PABP is essential to this phase, as it interacts with the nuclear pore proteins or nucleoporins, and with the nuclear export receptor (Nuclear Export Signal Export Receptor). It also acts as a support to other protein complexes which facilitate the exit of the messenger RNAs towards the cytoplasm and the rough endoplasmic reticulum, therefore towards the ribosomes. For these viruses, the presence of the poly A tail, therefore of the nuclear PABPs, is even more important. The use of 3′-deoxyadenosine, eliminating the possibility of binding this protein, limits the protein synthesis of viral genes even more effectively, causing the degradation of the mRNAs directly in the nucleus. Furthermore, these viruses do not have the ability to vary the length of the poly A tail, another factor which makes the antiviral function of cordycepin faster.

The pharmaceutical formulations can be formulated in dosage forms for oral, buccal, aerosol, parenteral, rectal, or transdermal administration.

For oral administration, the pharmaceutical formulations can be found, for example, in the form of tablets or capsules, hard or soft, prepared in the conventional fashion with pharmaceutically acceptable excipients such as binding agents (e.g., pregelatinized cornstarch, or methylcellulose hydroxypropyl); filling agents (e.g., lactose, microcrystalline cellulose or calcium hydrogen phosphate); lubricants (e.g., magnesium stearate, talc or silica); disintegrants (e.g., potato starch or sodium starch glycolate); or inhibiting agents (e.g., sodium lauryl sulfate). The tablets can be coated with the methods well known in the art. The liquid preparations for oral administration can be, for example, in the form of solutions, syrups or suspensions, preparations for aerosol, or they can be freeze-dried products to be reconstituted, before use, with water or other suitable vehicles. Such liquid preparations can be prepared through conventional methods with pharmaceutically acceptable additives such as suspending agents (e.g., sorbitol syrup, cellulose derivatives, or edible hydrogenated fats); emulsifying agents (e.g., lecithin or acacia); non-aqueous vehicles (e.g., almond oil, oily esters, ethyl alcohol or fractionated vegetable oils); and preservatives (e.g., methyl- or propyl-p-hydroxybenzoates or sorbic acid). The preparation can also conveniently contain flavorings, dyes, and sweetening agents.

The preparations for oral administration can be formulated appropriately to allow the controlled release of the active constituent.

For buccal administration, the formulations can be in the form of tablets or pills formulated in the conventional fashion, adapted to an absorption at the level of the buccal mucosa. Typical buccal formulations are tablets for sub-lingual administration. Aerosol formulation.

The formulation of the invention can be formulated for parenteral administration by injection. The formulations for injections can be presented in the form of a dose divided between inhibitor and other active molecules, dose to be administered, for example in vials, with an added preservative. The compositions can appear in such a form as suspensions, solutions, or emulsions in oily or aqueous vehicles and can contain agents of the formulation such as suspension, stabilizing and/or dispersing agents. Alternatively, the active ingredient(s) can be found in the form of a powder to be reconstituted, before use, with a suitable vehicle, for example with sterile water.

The formulation of the invention can also be a rectal formulation such as suppositories or retention enemas, for example containing the basic components of the common suppositories such as cocoa butter or other glycerides.

The preferred formulations for the purposes of the present invention are oral and injectable and aerosol formulations.

In particular, oral forms will be preferred in the case of preventive therapy or in cases of mild or asymptomatic infections.

The injectable or aerosol forms will be preferred in the case of emergency therapy, as emergency intervention on patients in serious or critical conditions.

The oral formulations can comprise 3′-deoxyadenosine and/or inosine and optionally adenosine.

In preferred embodiments, the injection formulations will comprise the inhibitor of the adenosine receptors A1 and A3, in particular inosine, in a separate vial from that containing 3′-deoxyadenosine.

According to the present invention, the dose of 3′-deoxyadenosine suggested for administration to a man (with a body weight of about 70 kg) ranges between 3 mg and 15 mg or between 5 mg and 12 mg of 3′-deoxyadenosine per dose unit, if injection is the chosen administration route. For oral administration, higher doses may be used depending on age, weight, physical and pathological condition, and other factors affecting the patient. For example, for oral administration, the dose unit will preferably contain between 50 mg and 150 mg of 3′-deoxyadenosine. The dosage of the inhibitor, preferably inosine, and any antiviral will be calculated based on the weight ratios indicated above.

The dose unit can be administered, for example, 1 to 4 times a day. The dose will depend on the route chosen for administration. It should be considered that continuous administration may be necessary for the most serious patients as well as continuous dose variations as a function of the severity of the clinical condition to be treated. The exact dose and route of administration will ultimately be at the discretion of the attending physician.

The formulations according to the invention can be prepared according to conventional methods, such as those described in Remington's Pharmaceutical Sciences Handbook, Mack Pub. Co., N.Y., USA, edition 2012.

The invention further relates to a kit, in particular a kit for emergency intervention, comprising a first container, for example a vial, containing an inosine solution for injection, and a second container, preferably a vial, containing an injectable solution of 3′-deoxyadenosine.

The invention will now be further described by means of the following formulation examples.

### Formulation Examples

### Example 1—Sustained-Release Gastro-Resistant Tablet

| | | |
|---|---|---|
| Inosine | 130 | mg |
| 3′-deoxyadenosine | 80 | mg |
| Microcrystalline cellulose | 80 | mg |
| Glyceryl Dibehenate | 150 | mg |
| Polyvinylpyrrolidone | 30 | mg |
| Magnesium stearate | 5 | mg |
| Silicon dioxide | 5 | mg |
| Gastro-resistant coating | 40 | mg |

### Example 2—Acid-Resistant Hard Capsule

| | | |
|---|---|---|
| Inosine | 160 | mg |
| 3′-deoxyadenosine | 100 | mg |
| Magnesium stearate | 5 | mg |

Hypromellose acid-resistant capsule size “0”

### Example 3—Single-Dose Water Dispersible Granulated Powder Stick

| | | |
|---|---|---|
| Inosine | 200 | mg |
| 3′-deoxyadenosine | 120 | mg |
| Glyceryl dipalmitostearate | 200 | mg |
| Polyvinylpyrrolidone | 30 | mg |
| Sorbitol | 180 | mg |
| Corn dextrins | 800 | mg |
| Poloxamer 188 EP | 50 | mg |
| Polysorbate 80 | 15 | mg |
| Rebaudioside A (Stevia) E960 | 10 | mg |
| Natural flavoring | 20 | mg |
| Dimethyl polysiloxane | 3 | mg |

### Example 4—Single-Dose Water Dispersible Granulated Powder Stick

| | | |
|---|---|---|
| Inosine | 200 | mg |
| 3′-deoxyadenosine | 120 | mg |
| Glyceryl dipalmitostearate | 200 | mg |
| Polyvinylpyrrolidone | 30 | mg |
| Sorbitol | 180 | mg |
| Corn dextrins | 800 | mg |
| Poloxamer 188 EP | 50 | mg |
| Polysorbate 80 | 15 | mg |
| Rebaudioside A (Stevia) E960 | 10 | mg |
| Natural flavoring | 20 | mg |
| Dimethyl polysiloxane | 3 | mg |

### Example 5—Effervescent Tablet

| | | |
|---|---|---|
| Inosine | 180 | mg |
| 3′-deoxyadenosine | 90 | mg |
| Glyceryl dipalmitostearate | 250 | mg |
| Potassium Bicarbonate | 343 | mg |
| Anhydrous potassium carbonate | 108 | mg |
| Anhydrous Citric acid | 384 | mg |
| Corn dextrins | 400 | mg |
| Sorbitol | 200 | mg |
| Polysorbate 80 | 15 | mg |
| Rebaudioside A (Stevia) E960 | 10 | mg |
| Natural flavoring | 20 | mg |
| Dimethyl polysiloxane | 3 | mg |

### Example 6—Single-Dose Sterile Solution for Intramuscular Injection

| Vial 1: | | |
|---|---|---|
| Inosine | 15 | mg |
| Sodium chloride | 18 | mg |
| Monobasic potassium phosphate | 0.5 | mg |
| Water | 1920 | mg |
| Sodium hydrate solution 25% | as needed to pH 7.0 | |
| Vial 2: | | |
| Adenosine | 12 | mg |
| Sodium chloride | 15 | mg |
| Monobasic potassium phosphate | 0.5 | mg |
| Water | 1920 | mg |

Sodium hydrate solution 25% as needed to pH 7.0

### Example 7—Single-Dose Sterile Solution for Intramuscular Injection

| Vial 1: | | |
|---|---|---|
| Inosine | 15 | mg |
| Sodium chloride | 18 | mg |
| Monobasic potassium phosphate | 0.5 | mg |
| Water | 1920 | mg |
| Sodium hydrate solution 25% | as needed to pH 7.0 | |
| Vial 2: | | |
| 3′-deoxyadenosine | 12 | mg |
| Adenosine | 8 | mg |
| Sodium chloride | 15 | mg |
| Monobasic potassium phosphate | 0.5 | mg |
| Water | 1920 | mg |

| | |
|---|---|
| Sodium hydrate solution 25% | as needed to pH 7.0. |

1. A method for treating a viral or bacterial syndrome wherein, for translation of the genes of the pathogens, messenger RNAs thereof are added with a poly A tail and therefore with related PABPs, and wherein partial or total absence of the protein or proteins reduces efficiency of a translation or protein synthesis of the genes thereof, to the point of preventing replication thereof, wherein the virus is a Coronavirus, comprising COVID-19, an orthomyxovirus (orthomyxoviridae), a picornavirus, poliovirus, or a togaviridae, and wherein the bacterium is malaria plasmodium comprising administering to a patient 3 -deoxyadenosine, configurational isomers, diastereoisomers, enantiomers thereof, racemes or mixtures thereof or salts with pharmaceutically acceptable acids, wherein, when 3 -deoxyadenosine is administered in combination with said inhibitor, the administration of3′-deoxyadenosine is subsequent or simultaneous to the administration of said inhibitor.

2. The method according to claim 1, wherein the 3′-deoxyadenosine is administered in combination with an agonist of the adenosine receptors A2a and/or A2b, w herein the administration of 3 deoxyadenosine is subsequent or simultaneous to the administration of said agonist.

3. The method according to claim 2, wherein the antagonistic inhibitor of the receptors A1 and A3 and the agonist of the adenosine receptors A₂ₐ and A_{2b} is inosine or hypoxanthine.

4. The method according to claim 3, wherein the 3′-deoxyadenosine is administered by injection and wherein the 3-deoxyadenosine is injected in a time between 5 minutes and 15 hours, from the administration of inosine or hypoxanthine.

5. The method according to claim 1, wherein the inhibitor and the 3′-deoxyadenosine are administered orally simultaneously or separately in rapid sequence, or in a single oral formulation.

6. The method according to claim 1, wherein the inosine is administered in a weight ratio between 1.2:1 and 2:1, with respect to 3 -deoxyadenosine.

7. The method according to claim 2, wherein 3^{f} -deoxyadenosine is administered separately or in conjunction with inosine and in combination with adenosine in patients of high severity.

8. A method for preparation of a medicine in the form of one or more separate dosage units, for treatment of viral infections from coronavirus comprising COVID-19, or an orthomyxovirus, the method comprising a step of adding in the medicine 3 -deoxyadenosine, in combination with at least one antagonistic inhibitor of the receptors A1 and A3 and agonist of the adenosine receptors A_{2A} and/or A_{2B}.

9. A formulation comprising 3 -deoxyadenosine, wherein said formulation is in the form of oral dosage and further comprises inosine.

10. The formulation according to claim 9, wherein the weight ratios are:
inosine between 1.2:1 and 2:1, with respect to 3′-deoxyadenosine.

11. A method of preventing or therapeutically treating a viral syndrome from Coronavirus comprising COVID-19, the method comprising administering to a patient inosine.

12. The method according to claim 1, in combination with at least one antagonistic inhibitor of the receptors A1 and A3 and possibly agonist of the adenosine receptors A_{2A} and/or A_{2B}, in preventive or therapeutic treatment of a viral syndrome from orthomyxovirus.

13. The method according to claim 1, in combination with at least one antagonistic inhibitor of the receptors A1 and A3 and possibly agonist of the adenosine receptors A2_{A} and/or A2_{B}, in preventive or therapeutic treatment of a viral syndrome from COVID-19.

14. The method according to claim 13, wherein the at least one antagonistic inhibitor comprises inosine.

15. The method according to claim 3, wherein the 3-deoxyadenosine is administered by injection and wherein the 3′-deoxyadenosine is injected in a time between 10 minutes and 6 hours, from the administration of inosine or hypoxanthine.

16. The method according to claim 8, wherein the at least one antagonistic inhibitor comprises inosine.

17. The method according to claim 11, wherein the inosine is administered individually or in combination, separately or jointly, with adenosine.

18. The method according to claim 12, wherein the at least one antagonistic inhibitor comprises inosine.

19. The method according to claim 1, wherein the inosine is administered in a weight ratio between 1.2:1 and 1.7:1, with respect to 3 -deoxy adenosine.

20. The formulation according to claim 9, wherein the weight ratio is inosine between 1.2:1 and 1.7:1, with respect to 3′-deoxyadenosine.
